(19) United States
(12) Patent Application Publication
(10) Pub. No.: US 2025/0213792 A1
(43) Pub. Date: Jul. 3, 2025

(54) Automatic Injection Device

(71) Applicant: Becton Dickinson France, Le Pont-de-Claix (FR)

(72) Inventors: Franck Carrel, Saint Jean de Vaulx (FR); Anne Guirimand, Sassenage (FR)

(21) Appl. No.: 18/850,515

(22) PCT Filed: Mar. 24, 2023
(86) PCT No.: PCT/EP2023/057580

§ 371 Date: Sep. 24, 2024

(30) Foreign Application Priority Data
Mar. 25, 2022 (EP) 22305378.6

(51) Int. Cl. A61M 5/20; A61M 5/315; A61M 5/32

(52) U.S. Cl. CPC A61M 5/2033; A61M 5/31501; A61M 5/3245; A61M 2005/206; A61M 2005/208; A61M 2005/31508; A61M 2005/3247; A61M 2205/276; A61M 2205/586; A61M 2205/8281

(57) ABSTRACT

The present disclosure relates to an automatic injection device including a body; a medical container including a barrel containing a product for injection, a distal tip provided with an injection needle and a stopper in sliding engagement within the barrel, the medical container being slidably arranged in the body between a proximal position and a distal position; a safety shield slidably mounted within the body between a distal initial position and a proximal second position; an outer shell enclosing the body, the outer shell being slidably movable in a distal direction relative to the body; a plunger rod slidably arranged in the body, the plunger rod including a proximal flange releasably locked in a proximal initial position by at least one flexible arm of the body, the plunger rod being movable in a distal direction when the proximal flange is unlocked to engage the stopper with a distal tip of the plunger rod; a cam cooperating with the safety shield so that a translation of the safety shield from the distal safety position to the proximal second position causes the cam to rotate within the body to an unlocking position; and a button slidably mounted at a proximal end of the body. In the initial position, the button is prevented from moving distally by the cam and, when the cam is in the unlocking position, the button is allowed to move in the distal direction. The outer shell cooperates with the safety shield and the button such that, when a user pushes the outer shell onto a patient's skin to perform an injection, the safety shield causes the cam to move to the unlocking position so as to unlock the button and the outer shell further pushes the unlocked button in the distal direction so as to cause the at least one flexible arm of the body to deflect outwardly to release the proximal flange of the plunger rod, thereby allowing the plunger rod to move to the injection position.

### CROSS-REFERENCE TO RELATED APPLICATIONS

This application is the United States national phase of International Patent Application No. PCT/EP2023/057580 filed Mar. 24, 2023, and claims priority to European Patent Application No. 22305378.6 filed Mar. 25, 2022, the disclosures of which are hereby incorporated by reference in their entireties.

### BACKGROUND OF THE INVENTION

### Field of the Invention

The present invention relates to a medical device for automatic injection of a product in a safe way, especially in emergency situations.

### Description of Related Art

Some illnesses necessitate regular injections of drugs or products, for instance on a daily basis. In order to simplify the treatment, some self-injectors have been provided in order to allow the patient to perform the injection on his/her own.

Since the patient is usually neither a nurse nor an educated person in medical devices, such self-injectors must prove to be very simple to use and also very safe. In particular, the insertion of the needle must be performed at the right depth, the correct dose of product must be injected, that is to say a complete injection must be performed, and the injector must be deactivated after use before it is disposed of. Preferably, the needle should not be exposed, before and after use, in order to prevent any accidental needlestick injury.

Another important requirement of these self-injection devices is that they must not be able to be activated inadvertently, before the patient is ready to perform the injection, and in particular before the device is correctly applied at the right injection site.

Some automatic injection devices comprise a safety shield adapted to cover the needle tip except during injection, and a button to be pressed by the patient to trigger the injection.

The document EP 2 921 191 discloses such an automatic injection device.

Before use, the safety shield and the needle are covered by a cap and the button is locked in order to avoid any accidental activation. The device comprises a mechanism configured to unlock the button once the safety shield has been pushed sufficiently onto the patient's skin.

Thus, the injection requires three actions from the user:
(1) removing the cap;
(2) pushing firmly the device onto the patient's skin to unlock the button; and
(3) pressing the unlocked button to activate the device and trigger injection.

However, when a drug has to be administered to the patient in an emergency situation, this three-step injection process may be too long or complex. For example, if a patient suffers from anaphylaxis, adrenaline has to be administered very quickly in order to avoid patient's death.

Thus, there is a need for self-injection devices that could be activated in a minimized number of steps without deteriorating the safety of the device. In particular, as indicated above, the device must not be able to be activated inadvertently and it also must avoid any accidental needlestick injury.

### SUMMARY OF THE INVENTION

The present invention meets this need by proposing a device for automatic injection of a product into an injection site, comprising:
a body;
a medical container comprising a barrel containing a product for injection, a distal tip provided with an injection needle and a stopper in sliding engagement within the barrel, the medical container being slidably arranged in the body between a proximal position and a distal position;
a safety shield slidably mounted within the body between a distal initial position and a proximal second position;
an outer shell enclosing the body, the outer shell being slidably movable in a distal direction relative to the body;
a plunger rod slidably arranged in the body, the plunger rod comprising a proximal flange releasably locked in a proximal initial position by at least one flexible arm of the body, the plunger rod being movable in a distal direction when the proximal flange is unlocked to engage the stopper with a distal tip of the plunger rod;
a cam cooperating with the safety shield so that a translation of the safety shield from the distal safety position to the proximal second position causes the cam to rotate within the body to an unlocking position; and
a button slidably mounted at a proximal end of the body,
wherein, in the initial position, the button is prevented from moving distally by the cam and, when the cam is in the unlocking position, the button is allowed to move in the distal direction,
wherein the outer shell cooperates with the safety shield and the button such that, when a user pushes the outer shell onto a patient's skin to perform an injection, the safety shield causes the cam to move to the unlocking position so as to unlock the button and the outer shell further pushes the unlocked button in the distal direction so as to cause the at least one flexible arm of the body to deflect outwardly to release the proximal flange of the plunger rod, thereby allowing the plunger rod to move to the injection position.

Thanks to the cooperation between the cam and the medical container, and between the outer shell and the button, only two actions—which are: (1) removing the cap and (2) pressing the safety shield onto the patient's skin—are required from the user to fully perform the injection.

Thus, the ergonomics of the device is improved. Such improved ergonomics is in particular beneficial in emergency situations since it simplifies the operation of the device and may reduce the time required to inject the full dose to the patient.

In the present text, the distal end of a component or of a device is to be understood as meaning the end furthest from the user's hand and the proximal end is to be understood as meaning the end closest to the user's hand. Likewise, in the present text, the distal direction is to be understood as meaning the direction of injection, and the proximal direction is to be understood as meaning the opposite direction to the direction of injection.

In the present text, the term “axial” designates a direction parallel to the direction of injection (the direction of injection also corresponding to a longitudinal axis of the injection device) and the term “radial” designates a direction perpendicular to the direction of injection.

In preferred embodiments, taken alone or in combination if more appropriate:
each flexible arm comprises an inclined inner surface tapering inwardly in the distal direction and at least one plunger retainer arranged distally from said inclined surface, the proximal flange of the plunger rod being in abutment with the at least one retainer in the initial position;
the button comprises at least one inner protrusion extending in the distal direction, said at least one protrusion being proximally spaced from the inclined surface of a respective flexible arm in the initial position and wherein, in the activation position, said at least one inner protrusion engages the respective inclined surface to deflect the flexible arm outwardly;
the button comprises at least one leg extending distally and the cam comprises a proximal end provided with at least one recess, and wherein, in the initial position, the button is locked by the cam by abutment of each leg with the proximal end of the cam, said leg being angularly spaced from each recess and, in the unlocking position of the cam, each leg faces a respective recess, thereby allowing the button to move in the distal direction relative to the cam;
the outer shell is made of a material opaque to UV light;
the outer shell comprises at least one window allowing visualizing internal components of the injection device;
the outer shell comprises at least one proximal window allowing visualizing the product for injection in the initial position and at least one distal window allowing visualizing the stopper of the medical container at the end of the injection;
the injection device further comprises a cap removably connected to a distal end of the outer shell, the cap comprising two rigid arms extending in the proximal direction;
the rigid arms cover the at least one window when the cap is connected to the outer shell;
the safety shield comprises at least one lug received in a respective first groove of the cam so that a movement of the safety shield in the proximal direction causes the cam to rotate;
the first groove presents an inclined first branch and an axial second branch connected to the first branch at a proximal end, such that, in the initial position, the lug is located at the distal end of the first branch and moves towards the proximal end of the first branch when the safety shield moves from the initial position to the second position;
the plunger rod is urged in the distal direction by a first spring;
the safety shield is urged in the distal direction by a second spring;
the injection device further comprises a ring supporting the medical container, the ring comprising at least one finger projecting radially outwardly, the cam further comprising at least one second groove receiving the respective at least one finger, the at least one second groove comprising a proximal portion, a distal portion and an inclined portion connecting the proximal portion to the distal portion, wherein the ring is locked at the proximal end of the groove and moves along inclined portion to the distal portion when the plunger rod pushes the medical container from the initial position to the injection position;
the at least one finger engages an axial groove of the body;
an axial distance between the proximal portion and the distal portion of the second groove is equal to a penetration depth of the needle within the patient's body;
a translation of the ring in the distal direction causes the cam to rotate so that the lug of the safety shield engages the second branch of the first groove of the cam.

### BRIEF DESCRIPTION OF THE DRAWINGS

The terms Fig., Figs., Figure, and Figures are used interchangeably in the specification to refer to the corresponding figures in the drawings.

Further features and advantages of the invention will be disclosed in the following detailed description, based on the appended drawings, wherein:

FIG. 1 is a perspective view of the injection device;

FIG. 2 is an exploded view of the button, the body and the cap of the injection device;

FIG. 3 is an exploded view of the medical container and the needle shield of the injection device;

FIG. 4 is an exploded view of the plunger rod and the medical container;

FIG. 5 is a perspective view of the cap and the needle shield;

FIG. 6 is a perspective view of the medical container and the supporting ring;

FIG. 7 is a perspective view of the safety shield;

FIG. 8 is a perspective view of the cam;

FIG. 9 is a perspective view of the assembly of the safety shield, the cam and the ring;

FIG. 10 is an exploded view of the button and the cam;

FIG. 11 is an exploded view of the outer shell;

FIG. 12 is a partial perspective view of the outer shell;

FIG. 13 is a sectional view of the injection device before use;

FIG. 14 is a sectional view of the injection device in the initial position;

FIG. 15 is a sectional view of the injection device in the unlocking position;

FIG. 16 is a sectional view of the injection device at the end of the injection;

FIG. 17 shows, from the left to the right, side views of the injection device before use, in the initial position and at the end of the injection.

### DESCRIPTION OF THE INVENTION

Unless otherwise specified, the components of the injection device are represented in their initial position, before use of the injection device. However, as will be explained below, some of the components are movable in translation and/or in rotation relative to each other to perform the injection. Thus, the description will refer not only to the initial position of the components, but also to one or more operative positions or phases that are reached during the injection process.

To summarize, before use, the body of the injection device is closed by a cap which protects the inside of the injection device from the outside environment.

In a first step, the cap is removed. As a result, the injection device is in an initial position. In said initial position, exposure of the needle and injection of the product are prevented by the fact that the needle and the medical container containing the product for injection are housed within the body of the injection device and that the plunger rod is locked in translation in the body.

In a second step, to perform an injection, a user handles an outer shell enclosing the body of the injection device and pushes the outer shell to apply a safety shield onto the patient's skin at an injection site. The safety shield is thus caused to translate in the proximal direction, which causes a cam coupled to the safety shield to rotate until unlocking a button of the injection device. As a result, the button, which is pushed by the outer shell, is allowed to translate in the distal direction relative to the body to release a plunger rod. As a result, a medical container engaging the stopper is caused to translate in the distal direction by the plunger rod, thereby allowing the needle to pierce the patient's skin at the right injection depth. Further, the movement of the stopper in the medical container in the distal direction expels the product from the medical container into the patient's body.

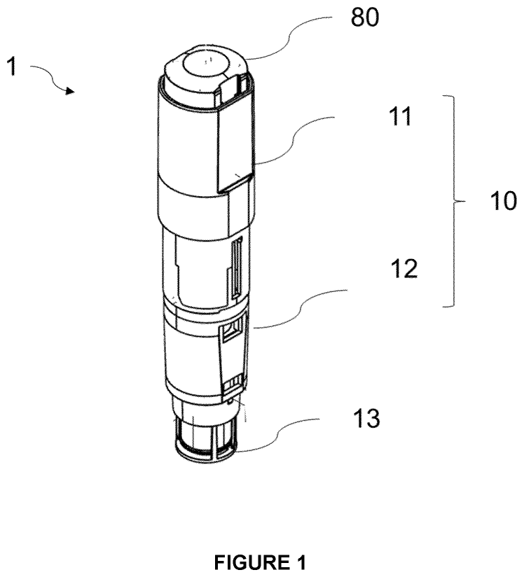

FIG. 1 shows a perspective view of a device for automatic injection according to an embodiment of the present disclosure and generally designated by reference number 1.

The device 1 comprises a housing 10 comprised of an upper body 11 and a lower body 12 that may be rigidly connected to each other.

The connection of the upper and lower bodies can be a snap-fit connection, screw-type connection, bayonet connection, or other means of connecting two parts together, in an unreleasable way or not. When the device is of a single use type, the means for connecting the upper body to the lower body are made unreachable to the user.

A medical container 20 such as, for example, a syringe, is received in at least one of the upper and lower bodies 11, 12.

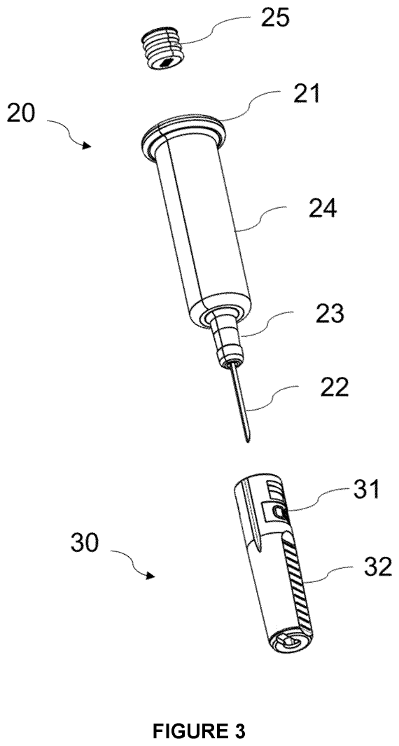

As shown in FIG. 3, the medical container 20 has a radial flange 21 defined at an open proximal end, and an injection needle 22 at a substantially closed distal end 23. Lateral walls extend between the proximal and distal ends and define a barrel 24 sized and shaped to contain a predetermined amount of a product for injection. The injection needle 22 may be fixed to the distal end 23, or removable therefrom, as a matter of design choice. The injection needle 22 is in fluid communication with the barrel 24 and provides an outlet port of the medical container 20 for the product.

A needle shield 30 is provided at the distal end of the medical container 20 to cover and protect the needle 22 before use of the device 1. The needle shield 30 also provides for a sealing means of the distal end 23 of the medical container before use. To that end, the needle shield 30 may comprise an inner elastomeric shield 31 sealingly engaging the distal end of the medical container and an outer rigid cap 32 surrounding the inner shield.

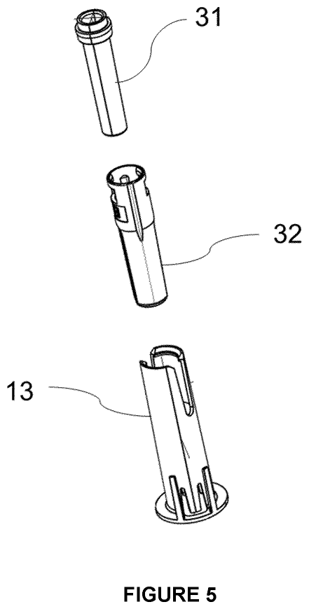

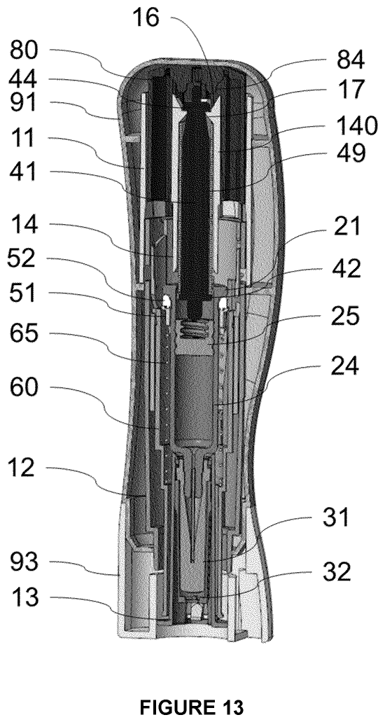

Before use (see FIGS. 5 and 13), a cap 13 engages the outer rigid cap 32 so that removal of the cap 13 simultaneously removes the inner elastomeric shield 31 and the outer rigid cap 32 from the distal tip of the medical container.

A stopper 25 is provided in the container 20 and is slidingly movable within the barrel 24. Movement of the stopper in the distal direction causes the product to be expelled from said medical container 20 through the injection needle 22 during the injection of the product into the patient. The stopper is typically made of an elastomeric material. The inner surface of the barrel and/or the outer surface of the stopper may be lubricated in order to reduce the gliding force of the stopper within the barrel.

A plunger rod 40 for causing the stopper to move with respect to the medical container 20, as will be explained later, is received within the proximal portion of the upper body 11.

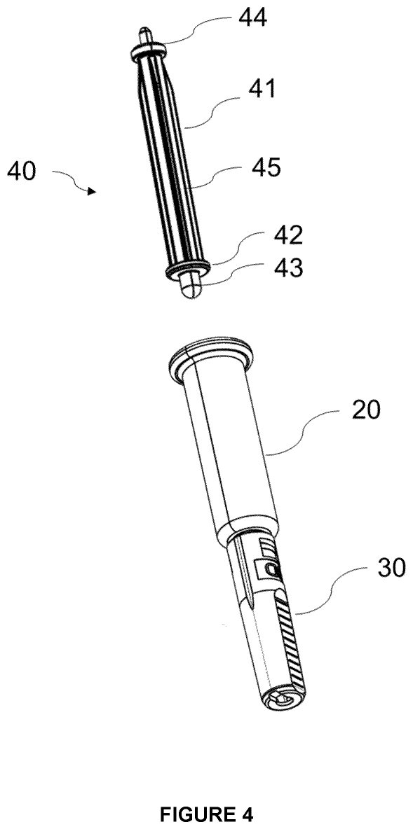

As shown in FIG. 4, the plunger rod 40 comprises a shaft 41 provided with a distal flange 42 and a distal tip 43 adapted to engage the stopper in the injection position. The shaft 41 also comprises a proximal flange 44. The shaft 41 may comprise one or more axial rib(s) designed for increasing its stiffness.

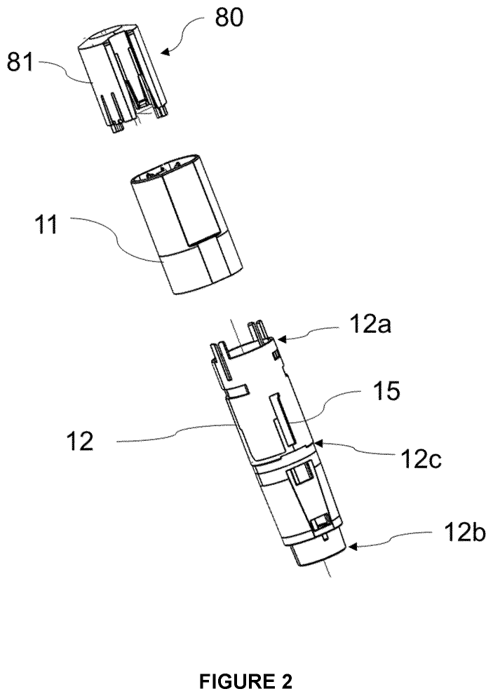

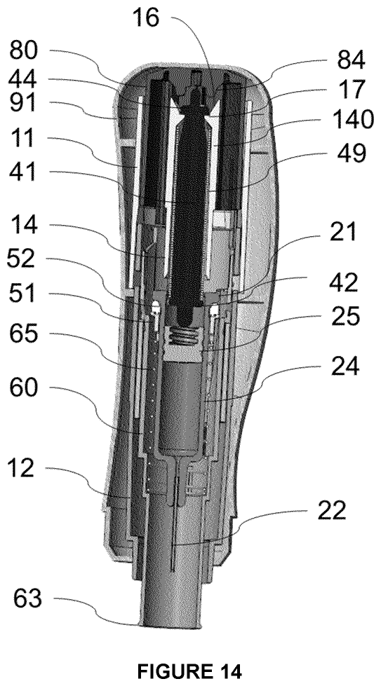

Returning to FIGS. 1 and 2, the upper body 11 has a generally cylindrical shape and is open at both ends. The distal end is connected to the lower body 12. The upper body further comprises an inner cylinder 14 receiving the plunger rod 40. The inner cylinder 14 comprises an axial wall parallel to the outer wall of the upper body and connected to said outer wall by a radial wall. The inner cylinder 14 has an open distal end and a proximal end comprising at least one flexible arm 140 comprising an inclined inner surface 16 tapering inwardly in the distal direction and at least one plunger retainer 17 arranged distally from said inclined surface 16. Preferably, the proximal end comprises two flexible arms 140 diametrically opposite to each other, or even two pairs of diametrically opposite flexible arms, with adjacent arms spaced by a 90° angle. For example, each plunger retainer 17 is in the form of a clip extending radially inwardly, comprising a proximal surface perpendicular to the direction of injection engaging the distal surface of the proximal flange 44 of the plunger rod in the initial position. Thus, in the initial position, the plunger rod 40 is prevented from moving distally by the plunger retainer(s) 17 (see FIG. 14). As will be explained in more detail below, the flexible arms 140 are designed to be able to deflect outwardly when a force is exerted onto the inclined surface 16 in the distal direction.

A button 80 is slidably mounted in the proximal portion of the upper body 11.

The button is prevented from moving in the proximal direction by clips 85 engaging the upper body 11, but is able to slide in the distal direction.

The button substantially surrounds the proximal end of the inner cylinder 14.

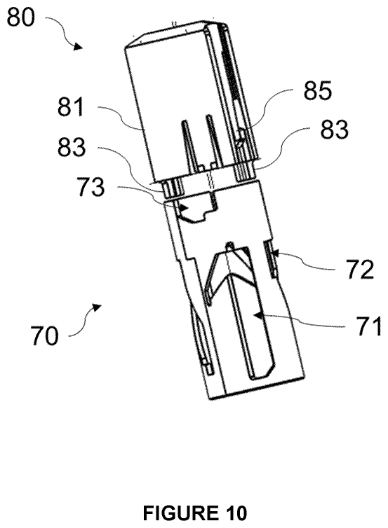

As shown in FIG. 10, the button 80 comprises an axial skirt 81 having a generally cylindrical shape open at its distal end and closed at its proximal end.

The button comprises at least one leg 83 extending distally from the skirt 81. Preferably, the button comprises two legs 83 diametrically opposite to each other.

The proximal end of the button comprises at least one inner protrusion 84 extending in the distal direction towards the flexible arms 140. In some embodiments, said protrusion may form a continuous annular rib. In other embodiments, at least two separate protrusions 84 are provided, each angularly aligned with a flexible arm 140, the number of protrusions 84 being equal to the number of flexible arms 140. In the initial position, said at least one protrusion 84 is proximally spaced from the inclined surface 16 of each flexible arm.

A first spring 49 (best seen in FIGS. 13 to 16) is arranged around the shaft 41 of the plunger rod, the distal end of the spring 49 bearing onto a proximal surface of the distal flange 42 and the proximal end of the spring 49 bearing onto a distal surface of the plunger retainers 17. In the initial position (see FIG. 14), the spring is in a compressed state. As a result, the spring 49 urges the plunger rod 40 in the distal direction. However, as noted above in the initial position, the plunger rod is locked by the plunger retainers 17 and thus is prevented from translating in the distal direction.

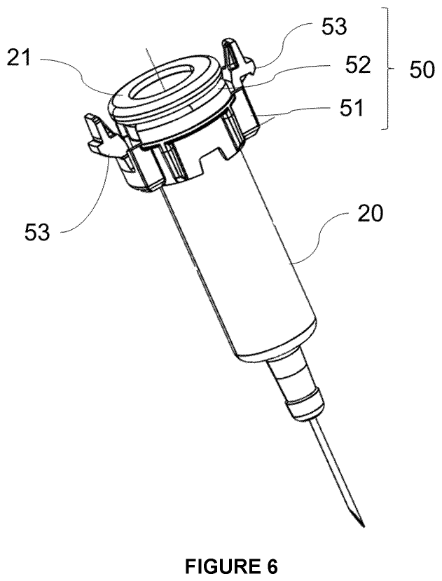

The medical container 20 is mounted in a supporting ring 50. As shown in FIG. 6, the ring may comprise a distal rigid portion 51 and a proximal elastomeric portion 52. The barrel of the medical container is inserted into the supporting ring 50 so that the proximal flange 21 bears onto the proximal surface of the proximal elastomeric portion 52. The medical container may be maintained in a fixed position relative to the ring by frictional engagement between the elastomeric portion 52 and the barrel.

The ring 50 comprises at least one finger 53 protruding radially from the rigid portion 51. Preferably, the ring comprises two fingers 53 diametrically opposite to each other.

The lower body forms a housing which receives at least partially the medical container 20 and the ring 50. As will appear later, the medical container 20 is movable relative to the lower body between an initial position, in which the tip of the needle does not extend beyond the distal end of the lower body, and an injection position, distally spaced relative to said initial position and in which the tip of the needle extends beyond the distal end of the lower body and is exposed over a predetermined length.

Returning to FIGS. 1 and 2, the lower body has a general cylindrical shape and is open at both ends. The lower body 12 has a distal part 12b and a proximal part 12a, the diameter of the proximal part 12a being greater than the diameter of the distal part 12b. The proximal part 12a and the distal part 12b are joined by a radial wall 12c. The proximal surface of the radial wall 12c forms an abutment surface for the ring 50.

The lower body comprises at least one axial groove 15 receiving a respective finger 53 of the ring 50. Preferably, the lower body has two axial grooves 15 diametrically opposite to each other, and the ring has two fingers, each received in a respective axial groove. As will be explained in more detail below, such axial groove serves as a guide for axial movement of the ring (and the container) relative to the lower body. Advantageously, said groove is located in a proximal portion of the lower body 12, which is covered by the upper body 11; thus, the groove 15 and the finger 53 are not accessible by a user from the outside of the injection device.

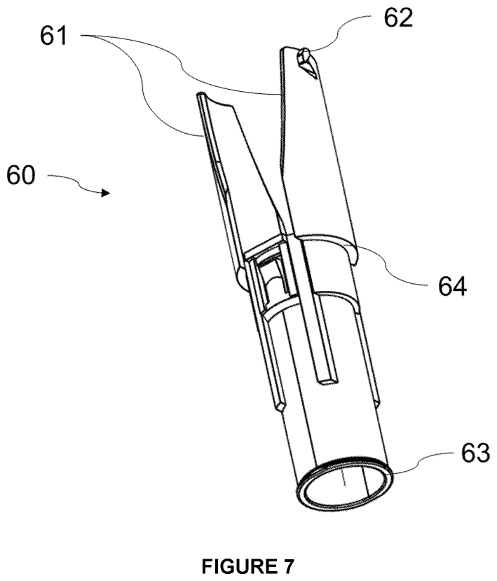

The injection device also includes a safety shield 60 that is at least partially received within a distal portion of the lower body. As shown in FIG. 7, the safety shield 60 comprises two opposite tongues 61 extending proximally. Each tongue 61 comprises a lug 62 extending radially outwardly.

The distal end of the safety shield 60 is advantageously provided with a radial flange 63 forming a bearing surface for applying the injection device onto the patient's skin, the width of the flange being chosen so as to distribute the pushing force on a sufficiently large surface for not injuring the patient.

The safety shield is coupled to a cam 70 pivotably mounted in the lower body 12.

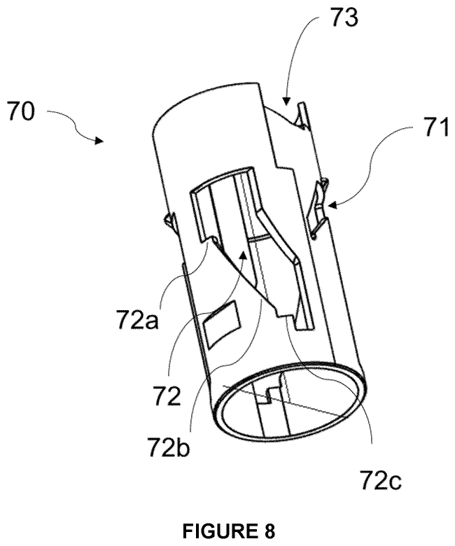

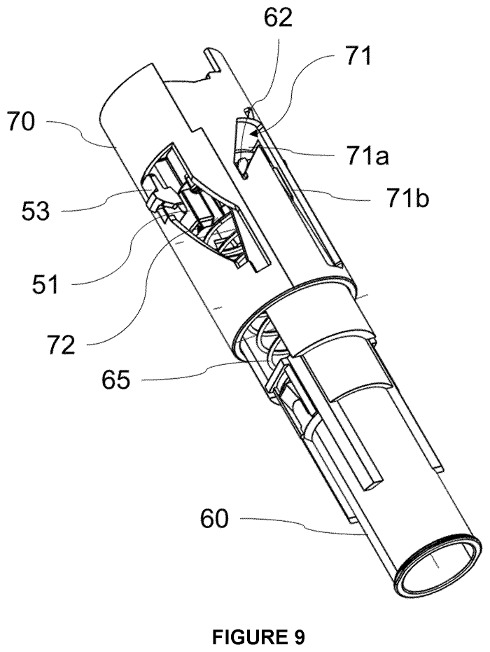

As shown in FIGS. 8 and 9, the cam 70 has a generally cylindrical shape and is open at both ends.

The cam 70 presents at least one first groove 71, preferably two first grooves diametrically opposite to each other.

Each first groove 71 comprises an inclined (i.e. non-axial) first branch 71a and an axial second branch 71b connected to the first branch at a proximal end of both branches 71a, 71b. Otherwise said, each first groove has the shape of number “1”.

Each lug 62 of the safety shield slidingly engages a respective first groove 71 in the cam 70.

In the initial position, the lug 62 is located at the distal end of the first branch 71a and moves towards the proximal end of the first branch 71a when the safety shield 60 moves from the initial position to the injection position. As a result, such a movement of the safety shield 60 in the proximal direction causes the cam to rotate by an angle depending on the slope of the first branch 71a and the stroke of the lug 62.

A second spring 65 is arranged between the proximal end of the rigid portion of the ring 50 and the flange 64 of the safety shield. In the initial position, the second spring 65 may be in a relaxed state, but a movement of the safety shield 60 in the proximal direction compresses the second spring 65. As a result, if the user releases the pressure applied onto the injection device, the second spring 65 urges the safety shield 60 in the distal direction.

The cam 70 further comprises at least one second groove 72 (preferably two second grooves 72 diametrically opposite to each other). Each second groove 72 comprises a proximal portion 72a extending perpendicular to the direction of injection and a distal portion 72c parallel to the proximal portion, and an inclined (i.e. non-axial) portion 72b connecting the proximal portion 72a to the distal portion 72c. Otherwise said, the proximal and distal portions 72a, 72c of each second groove are spaced both in the axially and angularly. The axial distance between the proximal portion 72a and the distal portion 72c of the second groove 72 is equal to the penetration depth of the needle within the patient's body.

The ring 50 is at least partially received within the cam with each finger 53 of the ring being in sliding engagement within a respective second groove 72.

In the initial position, each finger 53 of the ring is in the proximal portion 72a of the respective second groove 72.

The cam 70 further comprises at least one recess 73 (preferably two recesses 73 diametrically opposite to each other) formed in the proximal end of the cam. In the initial position, each leg 83 of the button is substantially in abutment with the proximal end of the cam, angularly spaced from the recesses 73 (see FIG. 10). As a result, in this initial position, the cam 70 prevents a translation of the button 80 in the distal direction. Even is the leg 83 is not in contact with the proximal end of the cam, the distance between the distal end of the leg 83 and the proximal end of the cam 70 is sufficiently small so that a translation of the button 80 in the distal direction would not allow the inner protrusion 84 to deflect the flexible arms 140 and thereby release the plunger rod 40.

When the cam 70 is caused to pivot by the movement of the safety shield 60 in the proximal direction along the first branch 71a of the first groove of the cam (as explained above), each recess 73 is caused to pivot relative a respective leg 83 of the button. The first groove 71 and the recess 73 are designed such that the leg 83 of the button faces a respective recess 73 (which corresponds to the unlocking position of the cam) when the lug 62 arrives at the connection between the first and second branches 71a, 71b of the first groove 71 (which corresponds to the second position of the safety shield). However, said rotation of the cam 70 does not cause any movement of the ring 50.

An outer shell 90 is provided around the body 10 and completely encloses the body. Advantageously, the outer shell may be formed of a plastic material opaque to UV light, in order to protect the product for injection. For example, the outer shell may be made of polycarbonate.

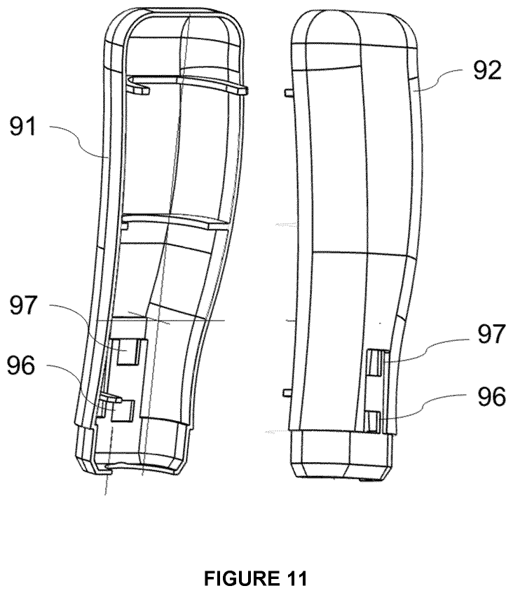

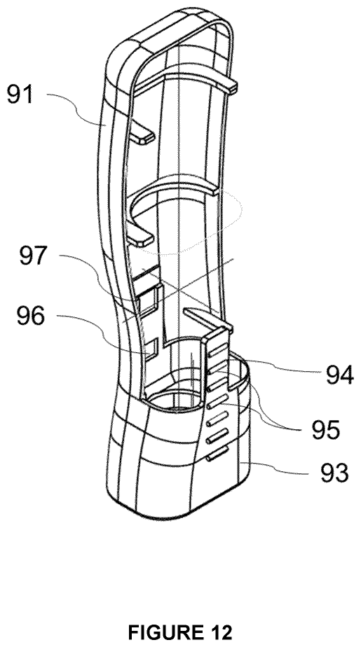

As shown in FIGS. 11 and 12, the outer shell 90 may comprise first and second half-shells 91, 92 that are substantially symmetrical relative to each other. The half-shells are rigidly connected to each other, for example by a snap-fit connection, or any other suitable means. The outer shell has an outer shape adapted to be held in a user's hand. In general, the outer shell 90 is intended to be gripped by the palm and the fingers of the user's hand.

The outer shell comprises a closed proximal end and an open distal end.

The outer shell 90 is provided with a cap 93 removably connected to the first and second half-shells 91, 92 to close the distal end before use of the injection device.

The cap 93 is rigidly coupled to the cap 13 so that, when the cap 93 is removed, the cap 13 and the needle shield 30 are also removed. In some embodiments (not illustrated), the cap 13 may be integral with the cap 93 so as to form a single piece.

The cap 93 comprises two arms 94 extending in the proximal direction. Each arm 94 may comprise, on its outer surface, a plurality of parallel ribs 95 substantially perpendicular to the direction of injection. Said ribs 95 may on the one hand form a gripping portion allowing a user to pull the cap in the distal direction, and on the other hand increase the stiffness of the arms, so as to avoid any twisting of the cap and the needle shield during removal.

Each half-shell 91, 92 advantageously comprises a proximal window 96 and a distal window 97. Before use of the injection device, the windows 96, 97 are masked by the arms 94 of the cap 93. Once the cap 93 has been removed, the windows 96, 97 function as a visual indicator by allowing a user to see internal components of the injection device, as will be explained in more detail below.

The outer shell 90 is slidably mounted relative to the body 10. The inner surface of the proximal end of the outer shell in in contact with the proximal surface of the button 80.

In the initial position, the distal end of the outer shell is located proximally relative to the distal end of the lower body 12. In said initial position, the protrusions 84 of the button are proximally spaced from the inclined surface 16 of each flexible arm of the inner cylinder of the body 10.

The functioning of the injection device 1 will now be explained.

The injection device is provided to a user ready-to-use, with the cap closing the distal end of the outer shell.

The medical container is filled with a predetermined dose of an injectable product—preferably a single dose thus providing a one-time use or disposable injection device.

Prior to use, the user removes the cap and the needle shield, without rotation of said needle shield, and places the safety shield against the patient's skin at an injection site. The patient may be the user or another person.

As the device is pressed against the patient's skin, the safety shield is caused to move in the proximal direction and into the lower body.

Due to the above-described safety features, the user cannot activate the device (i.e., cause the container to move from its initial position to its injection position and cause the plunger to push the stopper within the barrel) until the safety shield is caused to move a predetermined distance in the proximal direction so as to allow the cam to unlock the button.

When the device is pressed against the patient's skin via the outer shell (and the safety shield is moved out of its initial position in the proximal direction) until the button is unlocked, the device is automatically activated to begin an injection.

This unlocking of the button allows pressing the button with the outer shell in the distal direction and releasing the plunger rod, which is thus able to push the stopper in the distal direction. Said movement of the stopper thus causes a movement of the whole medical container in the distal direction from its initial position to its injection position, which also causes the needle to pierce the patient's skin.

Then, the plunger rod is still pushing the stopper in the distal direction in the barrel, which causes the injectable product to automatically be expelled from the container and into the patient's skin.

It is to be noted that, once the button has been unlocked, the unlocking of the plunger rod, the movement of the stopper and of the medical container happen during a continuous injection phase, without any further action from the user. In addition, the second position of the safety shield and the unlocking position of the cam are not stable positions but merely intermediate (temporary) positions reached during the triggering of the injection device.

Once the injection is complete, the user removes the device from the injection site and the safety shield is caused to automatically extend from the lower body to cover the now-contaminated tip of the needle. Advantageously, even if the user removes the device from the injection site before the injection is complete, the safety shield will automatically extend over the tip of the needle. Once the injection device is removed from the injection site and the safety shield is extended over the tip of the needle, the shield is locked in place and cannot thereafter be moved from its locked position in the proximal direction to expose the tip of the needle. The used injection device is thus rendered safe for handling and disposal.

The triggering of the injection device thus requires only one action from the user after the cap has been removed.

Referring to the figures, FIG. 13 illustrates the injection device before use. FIG. 14 illustrates the injection device in the initial position. When the user applies the injection device on the injection site by means of the bearing surface 63 of the safety shield 60 (the user holding the injection device via the outer shell 90), a distal force is exerted on the lower body 12 thereby causing the safety shield 60 to move relative to said lower body from the initial position, to a second position in which the cam unlocks the button, the second position of the safety shield being proximally spaced relative to said initial position.

During this proximal translation of the safety shield 60 to its second position, each lug 62 translates along the inclined branch 71a of the cam 70, thereby causing the cam 70 to rotate until the connection with the axial branch 71b, which corresponds to the unlocking position of the cam.

If the user releases the pushing force before reaching the second position, the compressed second spring 65 causes the safety shield to move back to the distal position in order to protect the needle.

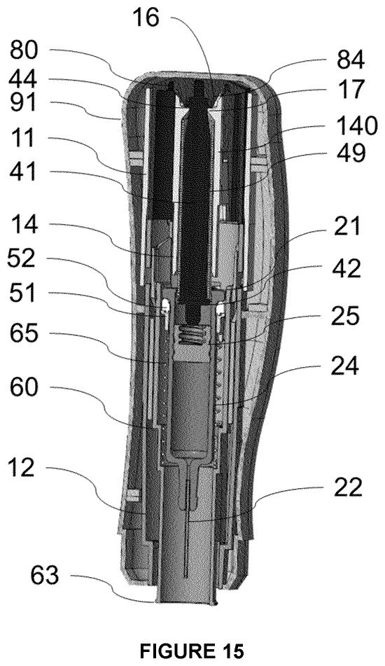

In the unlocking position of the cam, each leg 83 of the button 80 faces a respective recess 73 in the proximal end of the cam. As a result, if the user maintains the pushing force onto the outer shell 90, the outer shell will cause the button 80 to move distally relative to the body 10 until the legs 83 abut the proximal surface of the recesses 73 (see FIG. 15).

The axial dimensions of the recesses 73 and of the inner protrusion 84 are chosen so that, during this distal movement of the button, the inner protrusion 84 of the button engages the inclined surface 16, which causes each flexible arm of the inner cylinder 14 to deflect outwardly. Thus, the plunger retainers 17 disengage from the proximal flange 44 of the plunger rod.

As a result, the plunger rod 40 is caused to move in the distal direction under the distal force of the spring 49.

The force of the first spring 49 is greater than the force of the second spring 65. In particular, the force of the second spring 65 has to be sufficiently low to allow the safety shield 60 to move in the proximal direction in order to bring the cam in its unlocking position, so that the outer shell pushes the button that releases the first spring 49.

The distal end 43 of the plunger rod 40 engages the stopper and pushes it in the distal direction. The stopper which is in frictional engagement with the barrel thus causes the whole medical container to move in the distal direction.

Since the medical container is supported by the ring 50, the ring 50 is caused to translate in the distal direction with the medical container. During said translation, the fingers 53 of the ring 50 move within the second groove 72 of the cam. More precisely, each finger 53 engages the inclined portion 72b of the respective second groove 72, until the finger 53 reaches the distal portion 72c of the second groove, which prevents any further movement of the ring 50 and the medical container in the distal direction. Said translation of the ring 50 causes the cam 70 to further rotate, which allows the lug 62 to engage the second branch 71b of the first groove 71.

The medical container has thus reached its injection position. In this position, the needle protrudes from the distal end of the lower body 12 and pierces the patient's skin. As noted above, the axial stroke of the finger 53 between the proximal and distal portions 72a, 72c of the second groove 72 of the cam 70 defines the penetration depth of the needle. This ensures that the product be injected at the right depth between the patient's body.

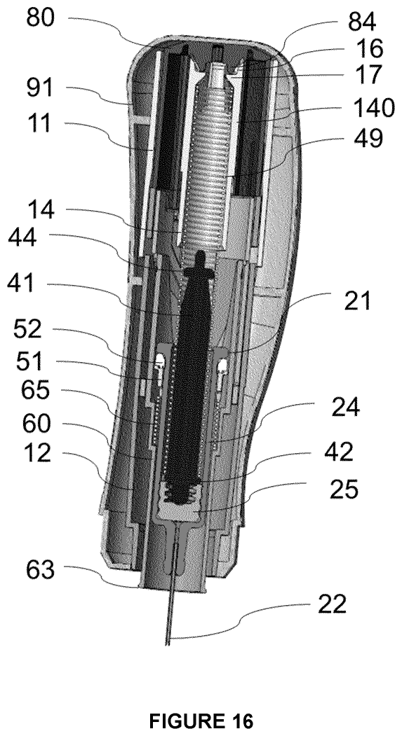

The plunger rod continues pushing the stopper to expel the product from the barrel through the needle until the stopper reaches the distal end of the barrel (see FIG. 16).

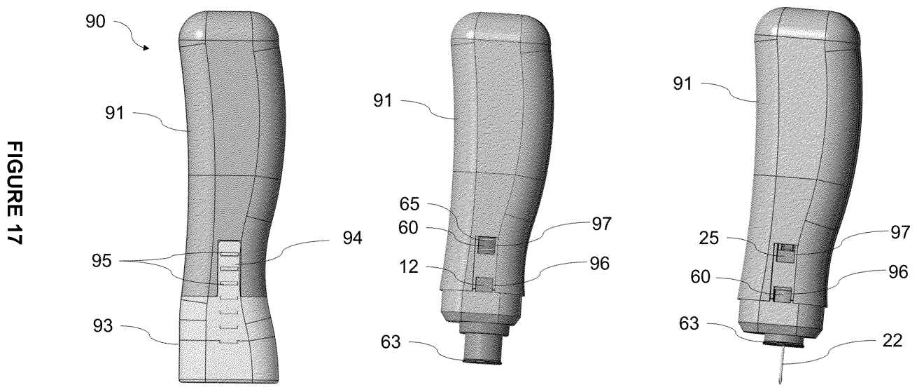

The windows 96, 97 formed in the half-shells allow visualizing the position of the internal components of the injection device at different stages of the injection process. For example, in the initial position (middle of FIG. 17), the user can visually inspect the product for injection through the proximal window 97 (the distal window 96 being obstructed by the lower body 12). In the injection position, the distal window 96 is unobstructed thanks to the relative movement of the outer shell and lower body 12. Thus, the user can visualize the stopper through the distal window 96 when the injection is completed (right side of FIG. 17).

Once the injection is completed, the user withdraws the injection device from the patient's skin.

This withdrawal causes the second spring 65 to push the safety shield 60 in the distal direction, the lugs 62 sliding within the second branch 71b of the first groove 71 of the cam 70, until a final position in which the safety shield covers and protects the needle. When in said final position, the safety shield is locked against proximal movement thereby preventing unintended access to the contaminated needle.

The injection device is thus very easy to use, even in emergency situations, and very safe since it prevents accidental needlestick injuries even in case said device is removed from the injection site before the injection of the product is actually completed.

1. An automatic injection device comprising:
a body;
a medical container comprising a barrel containing a product for injection, a distal tip provided with an injection needle and a stopper in sliding engagement within the barrel, the medical container being slidably arranged in the body between a proximal position and a distal position;
a safety shield slidably mounted within the body between a distal initial position and a proximal second position;
an outer shell enclosing the body, the outer shell being slidably movable in a distal direction relative to the body;
a plunger rod slidably arranged in the body, the plunger rod comprising a proximal flange releasably locked in a proximal initial position by at least one flexible arm of the body, the plunger rod being movable in a distal direction when the proximal flange is unlocked to engage the stopper with a distal tip of the plunger rod;
a cam cooperating with the safety shield so that a translation of the safety shield from the distal safety position to the proximal second position causes the cam to rotate within the body to an unlocking position; and
a button slidably mounted at a proximal end of the body
wherein, in the initial position, the button is prevented from moving distally by the cam and, when the cam is in the unlocking position, the button is allowed to move in the distal direction,
wherein the outer shell cooperates with the safety shield and the button such that, when a user pushes the outer shell onto a patient's skin to perform an injection, the safety shield causes the cam to move to the unlocking position so as to unlock the button and the outer shell further pushes the unlocked button in the distal direction so as to cause the at least one flexible arm of the body to deflect outwardly to release the proximal flange of the plunger rod, thereby allowing the plunger rod to move to the injection position.

2. The automatic injection device according to claim 1, wherein each flexible arm comprises an inclined inner surface tapering inwardly in the distal direction and at least one plunger retainer arranged distally from the inclined inner surface, the proximal flange of the plunger rod being in abutment with the at least one retainer in the initial position.

3. The automatic injection device according to claim 2, wherein the button comprises at least one inner protrusion extending in the distal direction, the at least one inner protrusion being proximally spaced from the inclined inner surface of a respective flexible arm in the initial position and wherein, in the activation position, said at least one inner protrusion engages the respective inclined inner surface to deflect the flexible arm outwardly.

4. The automatic injection device according to claim 1, wherein the button comprises at least one leg extending distally and the cam comprises a proximal end provided with at least one recess, and wherein, in the initial position, the button is locked by the cam by abutment of each leg with the proximal end of the cam, each leg being angularly spaced from each recess and, in the unlocking position of the cam, each leg faces a respective recess, thereby allowing the button to move in the distal direction relative to the cam.

5. The automatic injection device according to claim 1, wherein the outer shell is made of a material opaque to UV light.

6. The automatic injection device according to claim 1, wherein the outer shell comprises at least one window allowing visualizing internal components of the injection device.

7. The automatic injection device according to claim 6, wherein the outer shell comprises at least one proximal window allowing visualizing the product for injection in the initial position and at least one distal window allowing visualizing the stopper of the medical container at the end of the injection.

8. The automatic injection device according to claim 1, further comprising a cap removably connected to a distal end of the outer shell, the cap comprising two rigid arms extending in the proximal direction.

9. The automatic injection device according to claim 8, wherein the outer shell comprises at least one window allowing visualizing internal components of the injection device, and wherein the rigid arms cover the at least one window when the cap is connected to the outer shell.

10. The automatic injection device according to claim 1, wherein the safety shield comprises at least one lug received in a respective first groove of the cam so that a movement of the safety shield in the proximal direction causes the cam to rotate.

11. The automatic injection device according to claim 10, wherein the first groove presents an inclined first branch and an axial second branch connected to the first branch at a proximal end, such that, in the initial position, the lug is located at the distal end of the first branch and moves towards the proximal end of the first branch when the safety shield moves from the initial position to the second position.

12. The automatic injection device according to claim 1, wherein the plunger rod is urged in the distal direction by a first spring.

13. The automatic injection device according to claim 1, wherein the safety shield is urged in the distal direction by a second spring.

14. The automatic injection device according to claim 1, further comprising a ring supporting the medical container, the ring comprising at least one finger projecting radially outwardly, the cam further comprising at least one second groove receiving the respective at least one finger, the at least one second groove comprising a proximal portion, a distal portion and an inclined portion connecting the proximal portion to the distal portion, wherein the ring is locked at the proximal end of the groove and moves along inclined portion to the distal portion when the plunger rod pushes the medical container from the initial position to the injection position.

15. The automatic injection device according to claim 14, wherein the at least one finger engages an axial groove of the body.

16. The automatic injection device according to claim 14, wherein an axial distance between the proximal portion and the distal portion of the second groove is equal to a penetration depth of the needle within the patient's body.

17. The automatic injection device according to claim 11, further comprising a ring supporting the medical container, the ring comprising at least one finger projecting radially outwardly, the cam further comprising at least one second groove receiving the respective at least one finger, the at least one second groove comprising a proximal portion, a distal portion and an inclined portion connecting the proximal portion to the distal portion, wherein the ring is locked at the proximal end of the groove and moves along inclined portion to the distal portion when the plunger rod pushes the medical container from the initial position to the injection position, and wherein a translation of the ring in the distal direction causes the cam to rotate so that the lug of the safety shield engages the second branch of the first groove of the cam.
